(19) United States
(12) Patent Application Publication
(10) Pub. No.: US 2009/0186055 A1
(43) Pub. Date: Jul. 23, 2009

(54) MAKEUP COMPOSITIONS FOR KERATINOUS SUBSTANCES

(75) Inventors: Christophe Dumousseaux, Tokyo (JP); Makoto Kawamoto, Kawasaki (JP)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/302,863

(22) PCT Filed: Jun. 20, 2007
(86) PCT No.: PCT/IB07/52382

§ 371 Date: Feb. 11, 2009

Related U.S. Application Data
(60) Provisional application No. 60/841,557, filed on Sep. 1, 2006.

(30) Foreign Application Priority Data
Jun. 22, 2006 (FR) 06 05619

(51) Int. Cl. A61K 8/02

(52) U.S. Cl. 424/401

(57) ABSTRACT

The present invention provides a cosmetic composition comprising:
a physiologically acceptable medium comprising an aqueous phase; and
hollow monodisperse particles contained in the aqueous phase and capable of forming an ordered array of monodisperse particles after applying the composition to a support.

The present invention relates to cosmetic compositions, more particularly but not exclusively to those intended for making up keratinous substances, in particular the skin, lips, nails, eyelashes, and hair.

The use of pigments and colorants in makeup compositions is known.

However, the use of such pigments and colorants may lead to problems.

Thus, pigments and colorants may have relatively poor resistance to ultraviolet radiation and be spoiled by light.

Further, when the color is provided by an absorption phenomenon, the coloration produced may not be as clear and luminous as desired.

Finally, the range of pigments and colorants that can be used in cosmetics may prove to be insufficient.

The pigments and colorants may also impose restrictions on formulation.

To obtain a goniochromatic effect, it is known to use interference pigments. However, they are relatively complex and expensive to produce.

A goniochromatic effect present in the formulation may also be supplied by an ordered array of monodisperse particles, as disclosed in particular in International patent application WO-A-00/47167. Despite the fact that that publication is relatively old, the Applicant is currently not aware of any cosmetic product on the market that can produce a clear and luminous color for a period that is acceptable to the consumer by using an ordered array of monodisperse particles following application to keratinous substances.

The Applicant's International patent application WO-A-02/056854 discloses an iridescent composition for topical application, comprising at least one hydrosoluble surfactant and monodisperse particles in aqueous dispersion, said particles having a number average size of 50 nm [nanometer] to 300 nm and the quantity of said particles being at least 3% by weight relative to the total composition weight.

WO-A-05/018566 discloses a topical system for application to the skin, comprising a colloidal crystalline array in a hydrophilic phase and at least one phase containing an oil.

There is a need for further improvements to compositions that can produce a color by means of at least one ordered array of monodisperse particles, said array sometimes also being referred to as a “photonic crystal”.

In one aspects the invention provides a cosmetic composition comprising:
a physiologically acceptable medium comprising an aqueous phase;
hollow monodisperse particles contained in the aqueous phase and capable of forming an ordered array of monodisperse particles after applying the composition to a support.

In a further aspect, the invention provides a cosmetic composition comprising:
a physiologically acceptable medium comprising an anhydrous phase;
hollow monodisperse particles contained in the anhydrous phase and capable of forming an ordered array of monodisperse particles after applying the composition to a support.

In a further aspect, the invention provides a cosmetic composition comprising:
a physiologically acceptable medium;
hollow monodisperse particles with a wall thickness of 10 nm to 300 nm, capable of forming an ordered array of monodisperse particles after applying the composition to a support.

In a further aspect, the invention provides a cosmetic composition comprising:
a physiologically acceptable medium;
hollow monodisperse particles with a particle size of 800 nm to 3000 nm, capable of forming an ordered array of monodisperse particles after applying the composition to a support.

The term “anhydrous phase” denotes a phase that may contain less than 5%, in particular less than 3%, more particularly less than 2%, and still more particularly less than 1% of water relative to the weight of the phase.

The density of hollow particles is lower than that of solid particles and thus a greater volume is occupied for the same concentration by weight. When the monodisperse particles are constituted by a high density material, for example an inorganic material, using particles that are hollow can limit sedimentation in the composition.

The presence of air or another gas inside the particles after drying can result in a large difference in refractive index between the particles and the surrounding medium, which is favorable in terms of the intensity of the diffraction peak and thus to the development of a very intense coloration. Many non-volatile compounds may be added into or onto the composition without risking a loss of color and ending up with a transparent composition.

The mean particle size (external diameter) may be in the range 100 nm to 500 nm, preferably in the range 200 nm to 350 nm.

The wall size (difference between the outside diameter and the inside diameter) may be in the range 10 nm to 300 nm, preferably in the range 30 nm to 200 nm, more preferably in the range 50 nm to 150 nm. A very thin wall renders the particle mechanically fragile and more permeable to any surrounding liquid solvent. Thus, the wall preferably has no openings or holes.

In another embodiment the mean particle size may be in the range 800 to 3000 nm, preferably in the range 1200 to 2500 nm. Such a range of size enables to obtain a rainbow color effect due to the diffraction of the particle array after application. This range of size gives also a better compatibility with additional component in the formulation such as pigments or polymers.

The amount of the hollow monodisperse particles in the composition is advantageously 15%, more preferably 20% by weight. In particular, the amount in the phase containing them is 15% or more by weight.

A relatively high concentration of particles may facilitate the formation of a crystalline array, for example with the help of a cosmetic applicator. A relatively high concentration may result in pre-organization of the particles by electrostatic repulsion in the composition or after drying thereof.

The particles may form a compact crystalline array after application. The array may be discontinuous with the presence of fractures and dislocations.

An approximation for the wavelength λ of the light diffracted by the array is given by the Bragg relationship:

mλ=2nd sin θ;
where m is the diffraction order, n is the mean refractive index of the diffracting medium, d is the distance between two diffracting planes and θ is the Bragg angle between the incident light and the diffracting plane.

Thus, the diffracted wavelength depends mainly on the angle of observation and the distance between the particles. When the array formed is compact, that distance depends mainly on particle size. Thus, it is possible to obtain different goniochromatic colorations by varying the size of the particles present.

It is also possible to obtain reflection in the UV region (protection against UV) or in the IR region (heat reflective coating).

Since the distance between the particles varies during drying, it is relatively easy to obtain cosmetic compositions with a continuous variation (from red to blue) of color during drying following application, which entertains the consumer.

The invention may also allow a colored deposit to be formed following application of a composition that is initially colorless.

If desired, the invention can produce a cosmetic composition that is free of colorant or pigment, the color being produced by the ordered array of monodisperse particles.

The invention may also produce a colored deposit that is sensitive to an external stimulus such as temperature, humidity, or ultraviolet radiation.

Such a stimulus may exert an influence on the distance between the particles of the array and thus modify the color, as explained above.

The distance between the particles may be modified as a function, for example, of a variation in the dimension of the particles under the effect of an external stimulus and/or a variation in the distance between particles of substantially constant size due, for example, to a variation in the repulsive forces between them and/or a variation in the size of at least one compound present between the particles. The refractive index of the medium may possibly vary under the effect of an external stimulus, for example temperature.

The invention can produce a long-lasting, luminous coloration over a large surface area.

### Hollow Monodisperse Particles

The expression “hollow monodisperse particles” as used here denotes hollow particles with a mean size having a coefficient of variation, CV, of 15% or less.

The coefficient of variation, CV, is defined by the relationship

$$CV = \frac{s}{D} ,$$

s being the standard deviation of the particle size distribution and D the mean size thereof.

The mean size D and the standard deviation s of 250 particles may be measured by analyzing an image obtained using a scanning electron microscope, for example that with reference S-4 500 from HITACHI. Image analysis software may be used to facilitate this measurement, for example Winroof®, sold by Mitani Corporation.

Preferably, the coefficient of variation of monodisperse particles is 10% or less, more preferably 7% or less, or even 5% or less, for example substantially of the order of 3.5%. A low particle size dispersion can be favorable to the quality of the compact crystalline array formed and thus to the production of clear and brilliant colors.

In one embodiment the mean size D of the monodisperse particles may be in the range 80 nm to 800 nm, preferably in the range 100 nm to 500 nm, and may, for example, be selected as a function of the color or colors to be produced and of the surrounding medium.

A preferred range for the mean size is 150 nm to 450 nm, preferably 190 nm to 310 nm, to produce colors in the visible region. The mean size may be from 80 nm to 200 nm to filter UV.

The wall thickness may be in the range 10 nm to 300 nm, preferably in the range 30 nm to 200 nm, more preferably in the range 50 nm to 150 nm.

In one other embodiment the mean size D may be in the range 80 nm to 3000 nm, preferably in the range 1200 nm to 2500 nm, to produce raibow color effect. The wall thickness may be in the range 100 nm to 600 nm, preferably 200 nm to 500 nm.

In accordance with one aspect of the invention, the amount of monodisperse particles may, for example, be from 15% to 70%, preferably more than 20%, 25%, 30%, 35%, 40% or 45% by weight. Certain other aspects of the invention may employ other amounts, for example 1% to 70%.

The shape of the monodisperse particles must be compatible with forming an ordered array of monodisperse particles.

The array formed may be at least partially body centered cubic, face centered cubic, hexagonal close-packed or hybrid, formed starting from these arrangements, or otherwise.

Various examples of forming a crystalline array from monodisperse particles are given in the publication by Xia et al, Adv. Mater, 12, 693-713 (2000).

Preferably, the monodisperse particles are spherical in shape, but other forms are possible, especially those with axial symmetry.

The monodisperse particles may be single materials or composites.

The monodisperse particles may optionally be porous. The presence of small pores within the particles may reduce the refractive index of said particles.

The refractive index nₚ of the monodisperse particles is different from that, n_{c}, of the continuous medium extending around the particles after application of the formulation and the difference in said refractive indices is preferably 0.02 or more, more preferably 0.05 or more, still more preferably 0.1 or more, for example in the range 0.02 to 2, in particular in the range 0.05 to 1.

Too small a difference nₚ-n_{c} in the refractive index may require a large number of layers of particles of the ordered array to obtain the desired result. Too large a difference in the index may accentuate light diffusion by the layer and result in whitening of the deposit after application.

The refractive index of monodisperse particles is defined as the mean refractive index. For composite particles, it is calculated as a linear function of the proportion by volume of each component.

All monodisperse liquids corresponding to the same mean size D may have substantially the same refractive index.

The monodisperse particles may be colored, i.e. not white, for example to reinforce the intensity of the color produced and/or to avoid whitening of the composition after application to the keratinous substances.

One example of a colored particle used to form a colloidal crystal is given in the publication WO-A-05/012961.

The color of the monodisperse particles may be supplied by the choice of the material or materials constituting each monodisperse particle. It may enhance the absorption of light by the particles and reduce diffusion.

The monodisperse particles may incorporate at least one pigment or colorant, organic or inorganic, which may if necessary be fluorescent and fluoresce in the ultraviolet or infrared.

The monodisperse particles may include an inorganic compound, or it may even be entirely mineral.

When the monodisperse particles are inorganic they may, for example, comprise at least one oxide, especially metallic, and selected, for example, from oxides of silicon, iron, titanium, aluminum, chromium, zinc, copper, zirconium, and cerium, and mixtures thereof. The monodisperse particles may also include a metal, in particular titanium, silver, gold, aluminum, zinc, iron, copper, and their mixtures and alloys.

The monodisperse particles may comprise an organic compound, or even be entirely organic.

Examples of materials that may be suitable for producing the organic monodisperse particles and that may be mentioned include polymers, especially with a carbonaceous or silaceous chain, for example polystyrene (PS), polymethyl methacrylate (PMMA), polyacrylamide (PAM), and silicone polymers.

The monodisperse particles may include at least one polymer or copolymer that is capable of becoming ionized to improve dispersibility in the medium and provide electrostatic stabilization. In aqueous solution, that polymer or copolymer preferably contains carboxylic acid or sulfonic acid functions.

When the monodisperse particles are composites, they may, for example, comprise a hollow core and a shell produced from different substances, for example organic and/or mineral substances.

When the monodisperse particles are composites, the core material or shell may, for example, be selected to improve, for example, the stability of the monodisperse particles in the medium, to increase their refractive index and/or to color them and/or to provide fluorescence or magnetic susceptibility.

The core may be constituted by a material that is insoluble in the medium containing the particles, for example an inorganic material such as silica, for example, or an organic material such as an acrylic polymer, for example.

The shell may be constituted by polymeric chains that may be soluble in the medium containing the particles.

The monodisperse particle may be a “hairy” particle, comprising an insoluble hollow core and polymeric chains extending from the surface of the hollow core, especially by grafting.

Examples of “hairy” particles are given, for example, in the publication by Ishizu et al, Kagaku To Kogyo, 57(7) (2004) for a polymer core, or in the publication by Okubo et al, Colloid & Polymer Science, 280(3), pp 290-295 (2002) for a core of silica and a polymethyl methacrylate or poly(styrene/maleic anhydride) shell.

A further example of a “hairy” particle is given in the publication by Tsuji et al, Langmuir, 21, pp 2434-2437 (2005) for a core of polystyrene and a poly (N-isopropyl acrylamide) shell.

If appropriate, the presence of a core may allow encapsulation therein of a compound that should not come into direct contact with keratinous substances or the medium.

The composite monodisperse particles may also comprise inclusions of a first material in a matrix of a second material. As an example, the first material may have a high refractive index to increase the overall refractive index of the particle. The particle may, for example, include inclusions of nanoparticles, for example titanium oxide nanoparticles.

If appropriate, the monodisperse particles may have a dimension that is sensitive to an external stimulus, for example the concentration of a compound and/or temperature and/or pressure.

Examples of commercially available monodisperse particles that may be mentioned are SX866 particles sold by JSR.

### Hollow Particle Synthesis Methods

Monodisperse particles may be produced using synthesis methods as described, for example, in the publication by Xia et al, Adv. Mater, 12, 693-713 (2000), hereby incorporated by reference.

A conventional technique for producing hollow monodisperse particles consists in a first step of taking a monodisperse core then producing a shell with a different chemical composition. Dispersion polymerization is then carried out of styrene or acrylic monomers onto a silica core, for example. It is also possible to deposit silica onto a core of polystyrene by a sol-gel method. The second step consists of dissolving the core with a suitable solvent. A core of silica may be dissolved using an aqueous HF solution, or a core of PS may be dissolved using a solution of toluene. An example of such a synthesis is described in the publication by Xu et al, J. Am. Chem. Soc., 126(25), pp 7940-7945 (2004).

A further technique consists of carrying out emulsion polymerization in the presence of an organic solvent. The polymerizable monomers are soluble in that solvent but not the polymer. Thus, the result is the formation of particles in aqueous solution having a core constituted by an organic solvent. Said organic solvent may then be vacuum evaporated. By way of example, that technique is described in U.S. Pat. No. 4,908,271.

A further possible technique consists in carrying out emulsion polymerization of a core-shell latex with a core of a polymer that is capable of swelling under osmotic pressure (in an alkaline medium, for example). After swelling the core, the particles are dried to obtain hollow particles. Such a technique is described, for example, in the publication in Adv. Colloid interface Sci., 99(3), pp 181-213.

### Medium Containing the Hollow Monodisperse Particles

In accordance with the invention, at least before application, the hollow monodisperse particles may be contained in a physiologically acceptable medium to allow the formation of an ordered array of monodisperse particles on the support on which the composition is applied.

The term “physologically acceptable medium”, which is synonymous with the expression “cosmetically acceptable medium”, denotes a non-toxic medium that is capable of being applied to a support constituted by the keratinous substances of humans, in particular to the skin, mucosae, nails, and hair.

The physiologically acceptable medium is generally adapted to the nature of the support onto which the composition is to be applied and to the form in which the composition is intended to be packaged.

The hollow monodisperse particles may be contained in a liquid phase.

The medium containing the monodisperse particles may be entirely liquid or it may contain other particles, as appropriate.

The medium may be selected to be conducive to dispersing the particles in the medium before application thereof, to avoid particle aggregation.

The medium may be selected so that the ordered array of monodisperse particles is formed by regular stacking thereof, after application to the keratinous substances, the array not existing in the composition before application, and being formed, for example, during evaporation of a solvent contained in the composition.

As indicated above, the refractive index of the medium advantageously differs from that of the monodisperse particles, that difference in absolute terms preferably being 0.02 or more, more preferably 0.05 or more, in particular between 0.05 and 1, and more preferably 0.1 or more.

The medium may be aqueous, the monodisperse particles may be contained in an aqueous phase. The term “aqueous medium” denotes a medium that is liquid at ambient temperature and atmospheric pressure and that contains a large fraction of water relative to the total weight of the medium. The complementary fraction may contain or be constituted by physiologically acceptable organic solvents that are miscible with water, for example alcohols or alkylene glycols. The amount of water in the aqueous medium is preferably 30% or more by weight, more preferably 40%, still more preferably 50%.

The medium may be monophase or multiphase and may optionally include solids other than the monodisperse particles, especially finer or coarser particles.

Preferably, in the presence of solid bodies other than the monodisperse particles, the quantity of said bodies is sufficiently small so as to avoid interfering with the formation of the ordered array of monodisperse particles and the production of the desired result, in particular in terms of coloration.

The medium may comprise at least one compound having an OH bond, especially an alcohol function, in an amount that is, for example, 5% or more by weight, preferably 10%. Such a compound may slow down evaporation without disturbing the formation of the ordered array.

The medium may comprise an alcohol, such as ethanol or isopropanol, for example, or a glycol derivative, especially ethylene glycol or propylene glycol.

Preferably, the medium has a relative dielectric constant ∈ of 10 or more, more preferably 20 or more, still more preferably 30 or more. The dielectric constant is measured at a temperature of 25° C. A relatively high dielectric constant encourages ordering of the monodisperse particles into an array.

The conductivity of the composition may be in the range 5 μS·cm⁻¹ [microsiemen/centimeter] to 2000 μS·cm⁻¹, in particular in the range 10 μS·cm⁻¹ to 4000 μS·cm⁻¹, or even in the range 20 μS·cm⁻¹ to 400 μS·cm⁻¹.

The medium may be transparent or translucent, and may optionally be colored. The medium containing the monodisperse particles does not need to contain a pigment or colorant. The coloration of the medium may correspond to addition of an additional coloring agent.

The color of the medium corresponds, for example, to one of the colors that are capable of being generated by the ordered array of monodisperse particles, for example the color produced by the array when observed under normal incidence.

The medium may also be black in color, to limit light diffusion.

The ordered array of monodisperse particles may relatively easily produce the colors green, red, or blue. The color palette may be extended by the presence of an additional coloring agent, for example a colorant, an absorbing pigment or an effect pigment, for example in a concentration of 0.1% to 15% by weight.

The term “effect pigment” means, inter alia, reflective particles, nacres, goniochromatic coloring agents, or diffracting pigments, as defined below.

The presence of relatively large sized pigments such as nacres, for example, does not necessarily impede formation of the array by the pigment particles, but in contrast can encourage its formation by improving confinement of the hollow monodisperse particles, the large particles possibly being inserted into certain dislocations in the array.

The medium may thus include coarser particles with a size that is at least 3 times and preferably 5 times greater than that of the monodisperse particles, or even more, preferably 10 times greater.

These coarse particles may be particles of a pigment or a non coloring filler. The medium may thus include at least one effect pigment.

The presence of hollow monodisperse particles means that a periodic array can be produced after application to keratinous substances. Said array can produce a colored effect by light diffraction, and the Applicant has discovered that it is possible to combine it with a second optical effect using an effect pigment, while retaining the periodic array. The two optical effects are additive and the presence of pigment thus extends the color domain and the optical effects obtained by the array formed on the keratinous substances.

The effect pigment may be present in the formulation in a concentration in the range 0.1% to 70%, preferably 1% to 50%, more preferably 5% to 20%.

### Reflective Particles

Reflective particles can create highlights that are visible to the naked eye.

Reflective particles may have various forms. Said particles may be in the form of platelets or globules, in particular spherical. Said particles may comprise a substrate covered with a reflective material.

The substrate may be selected from glasses, metal oxides, aluminas, silicas, silicates, especially aluminosilicates and borosilicates, mica, synthetic mica, synthetic polymers, and mixtures thereof.

The reflective material may comprise a layer of metal or a metallic compound.

Particles of glass substrate coated with silver, in the form of platelets, are sold under the trade name METASHINE by Nippon Sheet Glass.

Examples of reflective particles that may be mentioned are particles comprising a substrate of synthetic mica coated with titanium dioxide or particles of glass coated with brown iron oxide, titanium oxide, tin oxide or a mixture thereof, such as those sold under the trade name REFLECKS® by ENGELHARD.

Pigments that are suitable for use in the invention are those from the METASHINE 1080R range sold by NIPPON SHEET GLASS CO. LTD. These pigments, more particularly those described in Japanese patent application JP-A-2001-11340, are C-GLASS glass flakes comprising 65% to 72% SiO₂ covered with a rutile (TiO₂) type titanium oxide layer. Said glass flakes have a mean thickness of 1 μm and a mean size of 80 μms, giving a mean size/thickness ratio of 80. They have blue, green or yellow glints or silver tints, depending on the thickness of the TiO₂ layer.

Particles with a dimension in the range 80 μm to 100 μm may also be mentioned, comprising a substrate of synthetic mica (fluorophlogopite) coated with titanium dioxide representing 12% of the total weight of the particle, sold under the trade name PROMINENCE by NIHON KOKEN.

The reflective particles may also be selected from particles formed by a stack of at least two layers with different refractive indices. Said layers may be polymeric or metallic in nature and in particular may include at least one polymeric layer. The reflective particles may be particles deriving from a multilayered polymeric film. Said particles have in particular been described in WO-A-99/36477, U.S. Pat. No. 6,299,979 and U.S. Pat. No. 6,387,498. Reflective particles comprising a stack of at least two layers of polymers are sold by 3M under the trade name MIRROR GLITTER. Said particles comprise layers of 2,6-PEN and polymethylmethacrylate in a ratio by weight of 80/20. Such particles are described in patent document U.S. Pat. No. 5,825,643.

### Nacres

The term “nacres” means colored particles of any shape, iridescent or otherwise, in particular produced in the shells of certain mollusks or synthesized, and that have a colored effect by optical interference.

Nacres may be selected from nacreous pigments such as mica titanium covered with an iron oxide, mica covered with bismuth oxychloride, mica titanium covered with chromium oxide, mica titanium covered with an organic colorant especially of the type mentioned above, as well as nacreous pigments based on bismuth oxychloride. They may also be mica particles with at least two successive layers of metallic oxides and/or organic coloring materials superimposed on their surface.

Examples of nacres that may be mentioned are natural mica covered with titanium oxide, iron oxide, natural pigment or bismuth oxychloride.

Examples of nacres that are available that may be mentioned are Flamenco nacres sold by ENGELHARD and TIMIRON nacres sold by MERCK.

### Goniochromatic Coloring Agents

The goniochromatic coloring agents referred to in the context of the present invention exhibit a color change, also termed a “color flop”, as a function of the angle of observation, which change is greater than that which occurs with nacres.

The goniochromatic coloring agent may, for example, be selected from interferential multilayer structures and liquid crystal coloring agents.

Examples of symmetrical interferential multilayer pigments that may be used in compositions of the invention are: CHROMAFLAIR by FLEX; SICOPEARL by BASF; XIRONA pigments by MERCK (Darmstadt) and INFINITE COLORS pigments from SHISEIDO or COLOR RELIEF pigments from CCIC.

It is also possible to use goniochromatic coloring agents with a multilayer structure comprising alternating polymeric layers, for example of the polyethylene naphthalene and polyethylene terephthalate type. Such agents are described in WO-A-96/19347 and WO-A-99/36478.

Examples of pigments with a polymeric multilayer structure that may be mentioned are those sold by 3M under the trade name COLOR GLITTER or those sold by Venture Chemical under the trade name Micro Glitter Pearl.

Liquid crystal coloring agents comprise, for example, silicones or cellulose ethers onto which mesomorphous groups are grafted. Examples of goniochromatic liquid crystal particles that may be used are those sold by CHENIX and that sold under the trade name HELICONE® HC by SICPA.

The composition may also comprise dispersed goniochromatic fibers. The size of such fibers may, for example, be in the range 50 μm to 2 mm. Goniochromatic fibers with a polyethylene terephthalate/nylon-6 bi-layer structure are sold by TEIJIN under the trade names MORPHOTEX and MORPHOTONE.

### Diffracting Pigments

The term “diffracting pigment” means a pigment comprising a periodic motif constituting a diffraction grating. The distance between the periodic motifs is of the same order of magnitude as visible light, and so that pigment can diffract light and produce a rainbow effect, for example.

Such pigments are commercially available under the trade name SPECTRAFLAIR from JDS Uniphase Corporation.

Such pigments may also be produced using the methods disclosed in patent documents U.S. Pat. No. 6,818,051, U.S. Pat. No. 6,894,086 and EP-A-1 634 619. Those patents describe pigments constituted by a 3-dimensional array of silica particles similar to the structure of opals. Inverse opal structures may also be obtained and used.

The medium in which the ordered array of monodisperse particles is formed may optionally be evaporated off after applying the composition.

Preferably, the medium comprises a volatile solvent. The term “volatile solvent” as used in the invention means any liquid that can be evaporated off in contact with the skin, at ambient temperature and at atmospheric pressure.

The medium may be selected so that the composition contains at least 10%, or even at least 30% of volatile solvent.

The pH of the composition may be from 1 to 11, for example 3 to 9. The pH that is most suited to the formation of an array may depend on the nature of the monodisperse particles. A basic pH is preferred when the monodisperse particles are mineral particles, especially when comprising silica.

The medium may comprise smaller particles having a mean size D that is lower than that of the monodisperse particles, by a factor of at least 2, preferably at least 3, to allow their insertion into voids left between the monodisperse particles of the array.

These interstitial particles may be mineral or organic and may improve cohesion of the array or modify absorption of light by the layers of the array.

Examples of interstitial particles that may be mentioned are nanoparticles of titanium dioxide, silica, iron oxide, carbon black, with a mean size of 5 nm to 150 nm, for example 10 nm to 100 nm.

Other examples of interstitial particles that may be mentioned are particles of a polymer that has already been polymerized in the composition before applying it to the keratinous substances, the medium comprising a latex, for example.

If appropriate, the size of the interstitial particles may vary as a function of an external stimulus and/or the concentration of a compound in the medium. The interstitial particles may be hydroabsorbing. By way of example, the particle size may vary as a function of the concentration of water in the medium.

If appropriate, the size variation of the interstitial particles may act on the distance between the monodisperse particles and thus have an effect on the color produced by the array.

The medium may comprise at least one polymer that can improve the behavior of the array after it has formed.

That polymer is, for example, not completely polymerized and/or cross-linked in the composition before applying it and drying it.

When the medium contains a polymer that is not completely polymerized and/or cross-linked before applying the composition to the keratinous substances, cross-linking and/or polymerization may be carried out after applying the composition to the keratinous substances.

Polymerization and/or cross-linking may, for example, be carried out after forming the array of monodisperse particles or, in a variation, before formation and/or concomitant therewith.

The medium may comprise a film-forming polymer.

### Film-Forming Polymer

In the present invention, the term “film-forming polymer” means a polymer that can, by itself or in the presence of an auxiliary film-forming agent, form macroscopically continuous film that adheres to keratinous substances, preferably a cohesive film and more preferably a film having cohesion and mechanical properties that are such that said film may be isolated and manipulated in isolation, for example when said film is produced by casting onto a nonstick surface such as a Teflon or silicone surface.

The composition may comprise an aqueous phase and the film-forming polymer may be present in this aqueous phase. It is then preferably a polymer in dispersion or an amphiphilic or associative polymer.

The term “polymer in dispersion” means polymers that are insoluble in water and present in the form of particles of varying sizes. The polymer may optionally be cross-linked. The mean particle size is typically in the range 25 nm to 500 nm, preferably in the range 50 nm to 200 nm. The following aqueous polymers in dispersion may be used: Ultrasol 2075 from Ganz Chemical, Daitosol 5000AD from Daito Kasei, Avalure UR 450 from Noveon, DYNAMX from National Starch, Syntran 5760 from Interpolymer, Acusol OP 301 from Rohm & Haas and Neocryl A 1090 from Avecia.

Acrylic dispersions sold under the trade name Neocryl XK-90®, Neocryl A-1070®, Neocryl A-1090®, Neocryl BT-62®, Neocryl A-1079® and Neocryl A-523® by AVECIA-NEORESINS, Dow Latex 432® by DOW CHEMICAL, Daitosol 5000 AD® or Daitosol 5000 SJ® by DAITO KASEI KOGYO; Syntran 5760® by Interpolymer, Allianz OPT by ROHM & HAAS, aqueous dispersions of acrylic or styrene/acrylic polymers sold under the trade name JONCRYL® by JOHNSON POLYMER or aqueous dispersions of polyurethane sold under the trade names Neorez R-981® and Neorez R-974® by AVECIA-NEORESINS, Avalure UR-405®, Avalure UR-410®, Avalure UR-425®, Avalure UR-450®, Sancure 875®, Sancure 861®, Sancure 878® and Sancure 2060® by GOODRICH, Impranil 85® from BAYER, Aquamere H-1511® from HYDROMER; sulfopolyesters sold under the trade name Eastman AQ® by Eastman Chemical Products, vinyl dispersions such as Mexomere PAM® from CHIMEX and mixtures thereof, are other examples of aqueous dispersions of particles of hydrodispersible film-forming polymers.

The term “amphiphilic or associative polymers” means polymers comprising one or more hydrophilic portions that renders them partially soluble in water and one or more hydrophobic polymers via which the polymers associate or interact. The following associative polymers may be used: Nuvis FX1100 from Elementis, Aculyn 22, Aculyn 44, Aculyn 46 from Rohm & Haas, Viscophobe DB1000 from Amerchol. Diblock copolymers constituted by a hydrophilic block (polyacrylate, polyethylene glycol) and a hydrophobic block (polystyrene, polysiloxane), may also be used.

Polymers that are soluble in an aqueous phase containing hollow monodisperse particles should be avoided since they may cause aggregation of the monodisperse particles. The film-forming polymer may thus be insoluble in said phase.

The composition may comprise an oily phase and the film-forming polymer may be present in that oily phase. The polymer may then be in dispersion or in solution. NAD (non-aqueous dispersion) or microgel (for example KSG) type polymers may be used, as well as polymers of the PS-PA type or styrene based copolymers (Kraton, Regalite).

Examples of non-aqueous dispersions of lipodispersible film-forming polymer in the form of non-aqueous dispersions of polymer particles in one or more silicone and/or hydrocarbon oils that may be surface stabilized by at least one stabilizing agent, in particular a block, graft or random polymer, which may be mentioned are acrylic dispersions in isododecane such as Mexomere PAP® from CHIMEX, dispersions of polymers of a grafted ethylenic polymer, preferably acrylic, in a liquid fat phase, the ethylenic polymer advantageously being dispersed in the absence of additional stabilizer for the particle surface such as that described in WO-A-04/055081.

Examples of film-forming polymers for use in the composition of the present invention that may be mentioned are synthetic polymers, of the radical or polycondensate type, polymers of natural origin, and mixtures thereof.

The term “radical film-forming polymer” means a polymer obtained by polymerization of monomers with an unsaturated bond, in particular an ethylenically unsaturated bond, each monomer being capable of self-polymerization (in contrast to polycondensates).

The radical type film-forming polymers may in particular be polymers or copolymers, vinyl, in particular acrylic polymers.

Vinyl film-forming polymers may result from polymerizing monomers with an ethylenically unsaturated bond containing at least one acid group and/or esters of these acid monomers and/or amides of these acid monomers.

Examples of monomers carrying an acid group that may be used are α,β-ethylenically unsaturated carboxylic acids such as acrylic acid, methacrylic acid, crotonic acid, maleic acid, or itaconic acid. Preferably, (methacrylic acid and crotonic acid are used, more preferably (meth)acrylic acid.

The esters of acid monomers are advantageously selected from esters of (meth)acrylic acid (also termed (meth)acrylates), especially alkyl (meth)acrylates, in particular C₁-C₃₀ alkyl, preferably C₁-C₂₀, aryl (meth)acrylates, in particular C₆-C₁₀ aryl, and hydroxyalkyl (meth)acrylates, in particular C₂-C₆ hydroxyalkyl.

Alkyl (meth)acrylates that may be mentioned are methyl methacrylate, ethyl methacrylate, butyl methacrylate, isobutyl methacrylate, 2-ethylhexyl methacrylate, lauryl methacrylate, and cyclohexyl methacrylate.

Hydroxyalkyl (meth)acrylates that may be mentioned include hydroxyethyl acrylate, 2-hydroxypropyl acrylate, hydroxyethyl methacrylate, and 2-hydroxypropyl methacrylate.

Aryl (meth)acrylates that may be mentioned include benzyl acrylate and phenyl acrylate.

Particularly preferred (meth)acrylic acid esters are alkyl (meth)acrylates.

In accordance with the present invention, the alkyl group in the esters may be either fluorinated, or perfluorinated, i.e. part or all of the hydrogen atoms of the alkyl group are substituted with fluorine atoms.

Examples of amides of acid monomers that may be mentioned are (meth)acrylamides, especially N-alkyl (meth)acrylamides, in particular C₂-C₁₂ alkyl. N-alkyl (meth)acrylamides that may be mentioned include N-ethyl acrylamide, N-t-butyl acrylamide, N-t-octyl acrylamide, and N-undecylacrylamide.

Vinyl film-forming polymers may also be produced by homo polymerization or copolymerization of monomers selected from vinyl esters and styrene monomers. In particular, said monomers may be polymerized with acid monomers and/or their esters and/or their amides, such as those mentioned above.

Examples of vinyl esters that may be mentioned are vinyl acetate, vinyl neodecanoate, vinyl pivalate, vinyl benzoate, and vinyl t-butyl benzoate.

Styrene monomers that may be mentioned are styrene and alpha-methyl styrene.

Film-forming polycondensates that may be mentioned include polyurethanes, polyesters, polyester amides, polyamides, epoxyester resins, and polyureas.

The polyurethanes may be selected from anionic, cationic non-ionic and amphoteric polyurethanes, from polyurethane-acrylics, polyurethane-polyvinyl pyrrolidones, polyester-polyurethanes, polyether-polyurethanes, polyureas, polyurea-polyurethanes, and mixtures thereof.

The polyesters may be obtained, in known manner, by polycondensation of dibasic carboxylic acids with polyols, in particular diols.

The dibasic carboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexane dicarboxylic acid, 1,4-cyclohexanedicarboxylic acid, isophthalic acid, terephthalic acid, 2,5-norbornane dicarboxylic acid, diglycolic acid, thiodipropionic acid, 2,5-naphthalenedicarboxylic acid, 2,6-naphthalenedicarboxylic acid. Said dicarboxylic acid monomers may be used alone or in combination with at least two dicarboxylic acid monomers. Of these monomers, phthalic acid, isophthalic acid and terephthalic acid are preferred.

The diol may be selected from aliphatic, alicyclic and aromatic diols. Preferably, the diol is selected from: ethylene glycol, diethylene glycol, triethylene glycol, 1,3-propanediol, cyclohexane dimethanol and 4-butanediol. Other polyols that may be used are glycerol, pentaerythritol, sorbitol and trimethylol propane.

The polyester amides may be obtained in a manner analogous to the polyesters, by polycondensation of dibasic acids with diamines or amino alcohols. The diamine used may be ethylenediamine, hexamethylenediamine or meta- or para-phenylenediamine. The amino alcohol used may be monoethanolamine.

Further, the polyester may include at least one monomer carrying at least one —SO3N group, where M represents a hydrogen atom, a NH4+ ion or a metal ion such as Na+, Li+, K+, Mg2+, Ca2+, Cu2+, Fe2+, Fe3+. It is also possible to use a bifunctional aromatic monomer including said —SO3M. group.

The aromatic nucleus of the bifunctional aromatic monomer that also carries a —SO3N group as described above may, for example, be selected from benzene, naphthalene, anthracene, diphenyl, oxydiphenyl, sulfonyldiphenyl and methylenediphenyl nuclei. Examples of a bifunctional aromatic monomer that also carries an —SO3M group: sulfoisophtalic acid, sulfoterephtalic acid, sulfophthalic acid and 4-sulfonaphtalene-2,7-dicarboxylic acid.

In one example of a composition of the invention, the film-forming polymer may be a polymer dissolved in a liquid fatty phase comprising oils or organic solvents (the film-forming polymer is then known as a liposoluble polymer). Preferably, the liquid fatty phase comprises a volatile oil, possibly mixed with a non-volatile oil.

Examples of liposoluble polymers that may be mentioned are vinyl ester copolymers (the vinyl group being directly bonded to the oxygen atom of the ester group and the vinyl ester having a saturated, linear or branched hydrocarbon radical containing 1 to 19 carbon atoms, bonded to the carbonyl group of the ester group) and at least one other monomer that may be a vinyl ester (different from the vinyl ester already present), an α-olefin (containing 8 to 28 carbon atoms), an alkylvinylether (the alkyl group of which contains 2 to 18 carbon atoms) or an allyl or methallyl ester (containing a saturated, linear or branched hydrocarbon radical containing 1 to 19 carbon atoms, bonded to the ester group).

Said copolymers may be cross-linked using cross-linking agents that may either be of the vinyl type or of the allyl or methallyl type, such as tetraallyloxyethane, divinylbenzene, divinyl octanedioate, divinyl dodecanedioate or divinyl octadecanedioate.

Examples of such copolymers that may be mentioned are: vinyl acetate/allyl stearate, vinyl acetate/vinyl laurate, vinyl acetate/vinyl stearate, vinyl acetate/octadecene, vinyl acetate/octadecylvinylether, vinyl propionate/allyl laurate, vinyl propionate/vinyl laurate, vinyl stearate/1-octadecene, vinyl acetate/1-dodecene, vinyl stearate/ethylvinylether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl propionate/vinyl stearate, allyl dimethyl propionate/vinyl stearate, vinyl propionate/vinyl stearate, cross-linked with 0.2% of divinyl benzene, vinyl dimethyl propionate/vinyl laurate, cross-linked with 0.2% of divinyl benzene, vinyl acetate/octadecyl vinyl ether, cross-linked with 0.2% of tetraallyloxyethane, vinyl acetate/allyl stearate, cross-linked with 0.2% of divinyl benzene, vinyl acetate/octadecene-1 cross-linked with 0.2% of divinyl benzene and allyl propionate/allyl stearate cross-linked with 0.2% of divinyl benzene.

Examples of liposoluble film-forming polymers that may be mentioned are copolymers of a vinyl ester and at least one other monomer that may be a vinyl ester, especially vinyl deodecanoate, vinyl benzoate or vinyl t-butyl benzoate, an α-olefin, an alkylvinylether or an allyl or methallyl ester.

Further examples of liposoluble film-forming polymers that may be mentioned are liposoluble copolymers, in particular those resulting from copolymerizing vinyl esters containing 9 to 22 carbon atoms or alkyl acrylates or methacrylates, the alkyl radicals containing 10 to 20 carbon atoms.

Such liposoluble copolymers may be selected from copolymers of vinyl polystearate, vinyl polystearate cross-linked with divinyl benzene, diallylether or diallyl phthalate, copolymers of stearyl poly(meth)acrylate, vinyl polylaurate or lauryl poly(meth)acrylate, said poly(meth)acrylates possibly being cross-linked with ethylene glycol dimethacrylate or glycol tetraethylene.

The liposoluble copolymers defined above are known and have been described in French patent application FR-A-2 232 303; they may have a mass average molecular weight of 2000 to 500000, preferably 4000 to 200000.

Examples of liposoluble film-forming polymers that may be used in the invention that may also be mentioned are polyalkylenes, in particular copolymers of C₂-C₂₀ alkenes such as polybutene, alkylcelluloses with a linear or branched, saturated or unsaturated C₁ to C₈ alkyl radical such as ethylcellulose or propylcellulose, copolymers of vinylpyrrolidone (VP) in particular copolymers of vinylpyrrolidone and C₂ to C₄₀ alkene, preferably C₃ to C₂₀. Examples of VP copolymers that can be used in the invention that may be mentioned are VP/vinyl acetate, VP/ethyl methacrylate, butylated polyvinylpyrolidone (PVP), VP/ethyl methacrylate/methacrylic acid, VP/eicosene, VP/hexadecene, VP/triacontene, VP/styrene and VP/acrylic acid/lauryl methacrylate copolymers.

Silicone resins, which are generally soluble or swellable in silicone oils may also be mentioned; they are cross-linked polyorganosiloxane polymers. The nomenclature of silicone resins is known as “MDTQ”, the resin being described as a function of the various monomeric siloxane units it includes, each of the letters “MDTQ” characterizing a type of unit.

Examples of commercially available polymethylsilsesquioxane resins that may be mentioned are those sold:
by Wacker with reference Resin MK, such as Belsil PMS MK;
by SHIN-ETSU with reference KR-220L.

Siloxysilicate resins that may be mentioned are trimethylsiloxysilicate (TMS) resins such as those sold with reference SR1000 by General Electric or with reference TMS 803 by Wacker. It is also possible to mention trimethylsiloxysilicate resins sold in a solvent such as cyclomethicone, and sold under the trade name “KF-7312J” by Shin-Etsu, or “DC 749”, “DC 593” by Dow Corning.

It is also possible to mention copolymers of silicone resins such as those mentioned above with polydimethylsiloxanes, such as the pressure-sensitive adhesive copolymers sold by Dow Corning with reference BIO-PSA and described in U.S. Pat. No. 5,162,410, or silicone copolymers derived from reacting a silicone resin as described above and a diorganosiloxane as described in the document WO-A-2004/073626.

In one implementation of the inventions the film-forming polymer is a linear ethylenic film-forming block polymer, which preferably comprises at least one first sequence and at least one second sequence having different glass transition temperatures (Tg), said first and second sequences being connected together via an intermediate sequence comprising at least one constituent monomer of the first sequence and at least one constituent monomer of the second sequence.

Advantageously, the first and second sequences and the block polymer are mutually incompatible.

Such polymers are described in documents EP-A-1 411 069 and WO-A-04/028488, for example.

The film-forming polymer may be selected from block or random copolymers and/or polymers including polyurethanes, polyacrylics, silicones, fluorinated polymers, butyl gums, ethylene copolymers, natural gums and polyvinyl alcohols and mixtures thereof. The monomers of the block or random copolymers comprise at least one association of monomers the resulting polymer from which has a glass transition temperature that is below ambient temperature (25° C.) and that may be selected from butadiene, ethylene, propylene, acrylic, methacrylic, isoprene, isobutene, a silicone, and mixtures thereof.

The film-forming polymer may also be present in the composition in the form of particles in dispersion in an aqueous phase or in a non-aqueous solvent phase, generally known as a latex or pseudolatex. Techniques for preparing such dispersions are well known to the skilled person.

The composition of the invention may comprise a plasticizing agent conducive to the formation of a film with the film-forming polymer. Such a plasticizing agent may be selected from any of the compounds known to the skilled person to be capable of fulfilling the desired function.

Clearly, this list of polymers is not exhaustive.

Preferably, when the medium containing the monodisperse particles contains a film-forming polymer, it is, for example, an aqueous dispersion of acrylic, vinyl, fluorinated or silicone polymer or mixtures thereof.

The amount of film-forming polymer(s) in the composition containing the monodisperse particles is from 0.1% to 10% by weight, for example.

When the composition containing the monodisperse particles contains a polymer that is not entirely polymerized and/or cross-linked, polymerization and/or cross-linking may be carried out by thermal initiation or by ultraviolet radiation.

Polymerization may also be carried out by adding an initiator and possibly a cross-linking agent.

When an array of monodisperse particles is to be produced in the medium, it is possible to add a monomer and an initiator and an optional cross-linking agent, then to carry out polymerization.

This may take place when manufacturing the formulation, or after application to the skin. This method allows polymers with a high molecular mass or cross-linked polymers to be produced. The rheology of the system formed can be varied in a bespoke manner.

The medium may also comprise a polymer allowing the formation of a gel, for example before or after applying the composition to the support to be made up.

### Gel Formation Polymers

Gel formation may, for example, improve the cohesion of the array of monodisperse particles and/or render it sensitive to an external stimulus and/or to the concentration of a compound in the medium, for example the concentration of water.

The gel formation polymer may be selected from derivatives of cellulose, alginates and their derivatives, especially their derivatives such as propylene glycol alginate, or their salts such as sodium alginate, calcium alginate, polyacrylic or polymethacrylic acid derivatives, polyacrylamide derivatives, polyvinylpyrrolidone derivatives, polyvinyl ether or alcohol derivatives, and mixtures thereof, inter alia.

In particular, the polymer may be selected from chemically modified cellulose derivatives, for example those selected from carboxymethylcellulose, sodium carboxymethylcellulose, carboxymethyl-hydroxyethylcellulose, carboxyethylcellulose, hydroxyethylcellulose, hydroxyethyl-ethylcellulose, hydroxypropylcellulose, hydroxypropylmethylcellulose, methylcellulose, sodium methylcellulose, microcrystalline cellulose, sodium cellulose sulfate and mixtures thereof.

The gel formation polymer may also be selected from natural polymeric derivatives such as gelatin and glucomannan and galactomannan polysaccharides extracted from grains, vegetable fibers, fruits, marine algae, starch, or plant resins, or it may be of microbial origin.

The quantity of gel formation polymer in the composition may be in the range 0.5% to 40% by weight, preferably in the range 1% to 20%.

The gel formation polymer may polymerize after applying the composition to the support to be made up. In a variation, the gel is formed before applying the composition to the keratinous substances, then applied thereto.

Hydrogels may be obtained from acrylamide, acrylic or vinylpyrrolidone monomers, for example, an example of a hydrogel obtained by this method based on N-isopropylacrylamide polymerized under a UV lamp in a colloidal crystal of polystyrene is, for example, described in patent WO-A-98/41859. The article by FOULGER et al, Advanced Materials, 13, 1898-1901 (2001) describes a hydrogel based on polyethylene glycol methacrylate and dimethacrylate.

The gel may also be produced before manufacturing the composition. It is possible, for example, to produce an oily gel based on a polydimethylsiloxane elastomer from an array of polystyrene spheres, as described in the article by H. Fudouzi et al, Langmuir, 19, 9653-9660 (2003).

### Fatty Phase

Although the composition containing the monodisperse particles may be free of oil, the composition of the invention may nevertheless include a fatty phase in some implementations. The hollow monodisperse particles may optionally be contained in that fatty phase.

The fatty phase may in particular be volatile.

The oil or oils may be introduced in a manner that does not lose the coloration effect or the desired spectral reflectance.

The composition may include an oil such as esters and synthesized esters, linear or branched hydrocarbons of mineral or synthetic origin, fatty alcohols containing 8 to 26 carbon atoms, fluorinated partially hydrocarbon and/or silicone oils, silicone oils such as polymethylsiloxanes (PDMS), volatile or otherwise with a linear or cyclic silicone chain, which are liquid or pasty at ambient temperature, and mixtures thereof; other examples are given below.

A composition in accordance with the invention may include at least one volatile oil.

### Volatile Oils

The term “volatile oil” as used in the present invention means an oil (or non-aqueous medium) that is capable of evaporating in contact with the skin in less than one hour, at ambient temperature and at atmospheric pressure.

The volatile oil is a volatile cosmetic oil, which is liquid at ambient temperature, which in particular has a non zero vapor pressure, at ambient temperature and at atmospheric pressure, in particular with a vapor pressure of 0.13 Pa [Pascal] to 40000 Pa (10⁻³ mm Hg [millimeters of mercury] to 300 mm Hg), in particular 1.3 Pa to 13000 Pa (0.01 mm Hg to 100 mm Hg), and more particularly 1.3 Pa to 1300 Pa (0.01 mm Hg to 10 mm Hg).

The volatile hydrocarbon oils may be selected from hydrocarbon oils of animal of plant origin containing 8 to 16 carbon atoms, in particular branched C₈-C₁₆ alkanes (also termed isoparaffins), such as isododecane (also denoted 2,2,4,4,6-pentamethylheptane), isodecane, isohexadecane, and, for example, oils sold under the trade names Isopars® or Permethyls®.

The volatile oils used may also be volatile silicones such as linear or cyclic volatile silicone oils, especially those with a viscosity of ≦8 centistokes (8×10⁻⁶ m²/s [square meter/second], especially containing 2 to 10 silicon atoms, in particular 2 to 7 silicon atoms, said silicones optionally comprising alkyl or alkoxy groups containing 1 to 10 carbon atoms. Examples of volatile silicone oils that may be used in the invention that may be mentioned are dimethicones with a viscosity of 5 and 6 cSt [centistokes], octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof.

It is also possible to use volatile fluorinated oils such as nonafluoromethoxybutane or perfluoromethylcyclopentane, and mixtures thereof.

It is also possible to use a mixture of the oils mentioned above.

### Non-Volatile Oils

The term “non-volatile oil” as used in the present invention means an oil with a vapor pressure of less than 0.13 Pa and in particular oils with a high molar mass.

The non-volatile oils may in particular be selected from hydrocarbon oils, that may if appropriate be fluorinated, and/or non-volatile silicone oils.

Examples of non-volatile hydrocarbon oils that may be suitable for the invention that may be mentioned are:
hydrocarbon oils of animal origin,
hydrocarbon oils of vegetable origin such as phytostearyl esters, such as phytostearyl oleate, phytsostearyl isostearate and lauroyl/octyldodecyle/phytostearyl glutanate, sold for example under the trade name ELDEW PS203 by AJINOMOTO, triglycerides constituted by esters of fatty acids and glycerol wherein the fatty acids may have chain lengths varying from C₄ to C₂₄, possibly being linear or branched, saturated or unsaturated; said oils are in particular heptanoic or octanoic triglycerides, wheat germ oil, sunflower seed oil, grape seed oil, sesame seed oil, corn oil, apricot kernel oil, castor oil, shea oil, avocado oil, olive oil, soya oil, sweet almond oil, palm oil, rapeseed oil, cottonseed oil, hazelnut oil, macadamia nut oil, jojoba oil, alfalfa oil, poppyseed oil, potimarron squash oil, gourd oil, blackcurrant oil, evening primrose oil, millet oil, barley oil, quinoa oil, rye oil, safflower oil, candlenut oil, passion flower oil, musk rose oil; shea butter or triglycerides of caprylic/capric acids such as those sold by STEARINERIES DUBOIS or those sold under the trade names MIGLYOL 810®, 812® and 818® by DYNAMIT NOBEL; and
hydrocarbon oils of mineral or synthetic origin, such as:
   synthesized ethers containing 10 to 40 carbon atoms;
   linear or branched hydrocarbons of mineral or synthetic origin, such as Vaseline, polydecenes, hydrogenated polyisobutene such as parleam, squalane and mixtures thereof, and in particular hydrogenated polyisobutene; and
   synthesized esters such as oils with formula R₁COOR₂ in which R₁ represents the residue of a linear or branched fatty acid containing 1 to 40 carbon atoms and R₂ represents a hydrocarbon chain, in particular branched, containing 1 to 40 carbon atoms provided that R₁+R₂ is ≧10.

The esters may in particular be selected from esters, especially of fatty acids, such as:
cetostearyl octanoate, isopropyl alcohol esters such as isopropyl myristate, isopropyl palmitate, ethyl palmitate, 2-ethyl-hexyle palmitate, isopropyl stearate or isostearate, isostearyl isostearate, octyl stearate, hydroxy esters such as isostearyl lactate, octyl hydroxystearate, diisopropyl adipate, heptanoates, in particular isostearyl heptanoate, octanoates, decanoates or ricinoleates of alcohols or polyalcohols such as propylene glycol dioctanoate, cetyl octanoate, tridecyl octanoate, 2-ethylhexyl 4-diheptanoate and palmitate, alkyl benzoate, polyethylene glycol diheptanoate, propyleneglycol 2-diethylhexanoate and mixtures thereof, benzoates of C₁₂ to C₁₅ alcohols, hexyl laurate, esters of neopentanoic acid such as isodecyl neopentanoate, isotridecyl neopentanoate, isostearyl neopentanoate, octyldodecyl neopentanoate, isononanoic acid esters such as isononyl isononanoate, isotridecyl isononanoate, octyl isononanoate, or hydroxyl esters such as isostearyl lactate, di-isostearyl malate;
esters of polyols, and esters of pentaetrythritol, such as dipentaerythritol tetrahydroxystearate/tetraisostearate;
esters of dimeric diols and dibasic acid dimers such as Lusplan DD-DA5® and Lusplan DD-DA7®, sold by NIPPON FINE CHEMICAL and described in patent application FR-03/02809,
fatty alcohols that are liquid at ambient temperature with a branched and/or unsaturated carbon chain containing 12 to 26 carbon atoms, such as 2-octyldodecanol, isostearyl alcohol, oleic alcohol, 2-hexyldecanol, 2-butyloctanol and 2-undecylpentadecanol;
higher fatty acids, such as oleic acid, linoleic acid, linolenic acid and mixtures thereof; and
di-alkyl carbonates, the 2 alkyl chains possibly being identical or different, such as dicaprylyl carbonate sold under the trade name Cetiol CC® by Cognis;
non-volatile silicone oils such as non-volatile polydimethylsiloxanes (PDMS), polydimethylsiloxanes comprising pendant and/or end alkyl or alkoxy groups on the silicone chain, groups each containing 2 to 24 carbon atoms, phenylated silicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, dimethicones or phenyltrimethicones with a viscosity of 100 Cst or less, and mixtures thereof; and
mixtures thereof.

The composition containing the monodisperse particles may be oil-free, and in particular contain no non-volatile oil.

### Kits

The invention also pertains to kits comprising a composition in accordance with the invention.

These kits may comprise at least one composition for forming a “base coat” and/or a “top coat”.

Thus, the kit may comprise:
a first composition comprising:
   hollow monodisperse particles; and
   a medium allowing the formation of an ordered array of monodisperse particles on a support onto which the composition is applied; and
a second composition comprising a film-forming polymer.

Said composition may allow the formation of a base coat or a top coat.

In a variation, the kit may also comprise:
a first composition comprising:
   hollow monodisperse particles; and
   a physiologically acceptable medium allowing the formation of an ordered array of monodisperse particles on a support onto which the composition is applied; and
a second composition comprising at least one coloring agent, for example a pigment or black colorant, or an effect pigment (reflective particles, nacres, goniochromatic coloring agent).

Said second composition may improve the optical properties of the first composition, for example.

The base coat and top coat may be present simultaneously, and the kit then comprises:
a first cosmetic composition comprising
   hollow monodisperse particles; and
   a physiologically acceptable medium allowing the formation of an ordered array of monodisperse particles on a support onto which the composition is applied;
a second cosmetic composition for application to a support before applying the first composition, to improve the adherence thereof to the support and to smooth the keratinous surfaces; and
a third cosmetic composition for application to the first composition to change its color and optionally improve the hold of the second composition.

### Base Coat

The base coat is compatible with its application to keratinous substances, for example the skin, lips, nails eyelashes or hair, depending on the nature of the makeup, in particular one of those listed above.

The base coat may comprise a polymer in particular selected from film-forming polymers.

Depending on the aspect of the invention, the base coat may have one or more of the following functions:
the base coat may smooth the support before applying the compound comprising the monodisperse particles to facilitate formation of the first layers of the array and obtain an array with mono crystalline zones that are as wide as possible;
the base coat may color the support to enhance or modify the color produced by the array. To this end, the base coat may comprise at least one coloring agent that can reduce the clarity of the support. The base coat may, for example, comprise a pigment or a black colorant or another color to create a colored background so that an additional color of a color given by the array of monodisperse particles can be added. Examples of colorants or pigments that may be present in the base coat that may be mentioned include black iron oxide, carbon black, and black titanium dioxide;
the base coat may improve the adherence of the composition containing the monodisperse particles on the made up support. To this end, the base coat may comprise at least one polymer with adhesive or pro-adhesive properties, i.e. capable of becoming adhesive by interaction with another compound. The polymer may in particular have adhesive or pro-adhesive properties as defined in patents FR-A-2 834 884, FR-A-2 811 546, and FR-A-2 811 547.

The base coat may also exert an action on the surface tension of the keratinous substances to allow, for example, good wettability by the layer of the composition containing the monodisperse particles and encourage stacking of the monodisperse particles.

The base coat may comprise one polymer carrying out at least two of the functions mentioned above, for example smoothing and increasing adherence, or even a coloring function.

The base coat may be formulated as a function of the nature of the monodisperse particles.

In the non limiting examples of implementations of the invention, the monodisperse particles may be formed from polystyrene and the base coat may comprise a non-aqueous dispersion, NAD, in isododecane or DAITOSOL (Daito Kasei) or ULTRASOL (Ganz Chemical) polymers. In other examples, the monodisperse particles are formed from silica, and the base coat may comprise an Eastman AQ (20%) or PVA (10%) polymer.

The base coat may include a volatile phase.

The polymer is preferably capable of forming a film after applying and drying the composition. Film formation may occur with the aid of a coalescence agent. The polymer may be in dispersion or in solution in an aqueous or anhydrous phase. Preferably, said polymer is in dispersion in water or in an oil. More preferably, the polymer contains at least one function that can ionize in aqueous solution, such as a carboxylic acid. The polymer is preferably insoluble in contact with an aqueous phase after application and drying.

It is also possible in this method to use monomers or pre polymers in the base coat that are also capable of polymerizing after applying to the skin, by the action of UV, heat or the presence of water, for example. Examples that may be mentioned are cyanoacrylate monomers or low mass silicone polymers carrying reactive functions.

Examples of polymers in aqueous dispersion that may be mentioned are: Ultrasol 2075 from Ganz Chemical, Daitosol 5000AD from Daito Kasei, Avalure UR 450 from Noveon, DYNAMX from National Starch, Syntran 5760 from Interpolymer, Acusol OP 301 from Rohm & Haas and Neocryl A 1090 from Avecia.

Examples of polymers in oily dispersion that may be mentioned are: NADs and the polymers disclosed in patent application EP-A-1 411 06 from l'Oréal, and the ACRIT 8HV-1023 acrylic-silicone polymer dispersion from Tasei Chemical Industries.

The volatile phase may be an aqueous phase or an anhydrous phase.

With an aqueous phase, it is preferably constituted by water, alcohol and glycol.

With an anhydrous phase, it is preferably constituted by at least one volatile oil as defined above.

The base coat may optionally be colored. With a colored base coat, it may contain colorants or pigments. The pigments are preferably dispersed as finely as possible to avoid making the film formed rough.

The base coat may contain other solid components (fillers, effect pigments) or other non-volatile liquid components. These are preferably be in small quantities.

### Top Coat

The top coat may, in addition to changing a visible characteristic such as color or brilliance, function as explained above to improve the hold of the array of monodisperse particles on the support, in particular to increase the frictional resistance of the array and prevent it from flaking off.

The top coat may comprise one or more polymers that may optionally penetrate into the array of particles, penetration of the polymer changing the refractive index of the medium surrounding the particles and thus changing the color.

The top coat may have a volatile phase, which could set a time limit on the color change, as it would strop during evaporation of the volatile phase.

The second composition may comprise a volatile oil as defined above.

The top coat may include a non-volatile solvent, which may increase the duration of the color change. Said solvent penetrates into and remains in the medium between the particles and modifies the refractive index around the particles.

The second composition for forming the top coat may thus comprise a non-volatile oil as defined above.

The top coat may be highly transparent to avoid affecting the color and/or intensity of the color deriving from the array of monodisperse particles.

The top coat may also be colored in order, for example, to exert an influence on the color and/or brilliance produced by the array of monodisperse particles.

The top coat may also slow down the uptake of moisture or drying out of the layer of the composition containing the ordered array and reduce variability of the result with time.

In contrast, the top coat may also increase the sensitivity to the environment, in order, for example, to create a dependency of the color on temperature or ambient humidity.

The top coat preferably includes a film-forming polymer.

The formulation for the top coat may be adapted to the nature of the monodisperse particles.

In the example of particles monodisperse in silica or polystyrene, the top coat may include a non-aqueous dispersion, NAD, in isododecane. When the monodisperse particles are formed from polystyrene, the top coat may, for example, comprise an acrylic copolymer or PVA. For polystyrene monodisperse particles, the top coat comprises, for example, a non-aqueous dispersion NAD, PVA (10%) or polymers from Eastman AQ (20%), DAITOSOL or ULTRASOL.

The top coat may contain monodisperse particles with a mean size that is different from that of the monodisperse particles covered by the top coat. This means that the color of the subjacent composition may be changed.

The top coat may then optionally be covered by a layer intended to improve the hold.

### Additives

The cosmetic composition containing the monodisperse particles, the base coat and the top coat may include at least one additive selected from the usual adjuvants in the cosmetic field, such as fillers, hydrophilic or lipophilic gelling agents, active ingredients, which may be hydrosoluble or liposoluble, preservatives, moisturizers such as polyols and in particular glycerin, sequestrating agents, antioxidants, solvents, fragrances, physical and chemical sunscreens, especially those screening UVA and/or UVB, odor absorbers, pH adjusters (acids or bases), and mixtures thereof.

In particular, the additive or additives may be selected from those mentioned in the CTFA Cosmetic Ingredient Handbook, 10^{th} Edition Cosmetic and fragrance Assn, Inc., Washington D.C. (2004), hereby incorporated by reference.

### Galenical Forms

The composition containing the hollow monodisperse particles may be presented in various galenical forms that are used in the cosmetic field, used for topical application: direct, reverse or multiple emulsions, gels, creams, solutions, suspensions or lotions.

The composition may be in the form of an aqueous solution or oily solution, in particular gelled, an emulsion with a liquid or semi-liquid consistency of the milk type obtained by dispersing a fatty phase in an aqueous phase (O/W) or vice versa (W/O), a triple emulsion (W/O/W) or O/W/O), or a suspension or emulsion with a soft consistency.

The composition of the invention may constitute a skin care composition, makeup composition and/or sun protection composition. For a sun protection composition, the particle size may be selected so as to reflect UVA and/or UVB wavelengths, the particle size being selected, for example, using the Bragg relationship, mλ=2nd sin θ, where m is the diffraction order, n is the mean refractive index of the medium, θ is the angle of incidence between the incident light and the diffraction planes, and d is the distance between the diffraction planes.

The composition may be in the form of a face makeup product, especially for the skin and/or lips, eyes or nails.

### Makeup Methods

The invention also provides a method of making up keratinous substances, comprising the following steps:
applying a base coat to a support to be made up; and
applying a cosmetic composition comprising hollow monodisperse particles and a medium allowing the formation of an ordered array of monodisperse particles to the base coat.

Said method can improve the quality of application of the composition comprising the monodisperse particles, in particular when they are in an aqueous medium, and can also obtain good “crystallization” after application to the skin or hair, for example.

As mentioned above, the base coat can control and even out the surface properties of the keratinous substances, in particular the surface tension. It can also smooth and even out surface roughness. An electrostatic repulsion effect may also occur if the base coat is capable of creating an electrostatic charge in contact with water.

Apart from very substantially improving the arrangement of the particles, the base coat may fix the layer of monodisperse particles, rendering it more stable to external attack.

In accordance with this method, the base coat preferably contains a polymer and a volatile phase.

The composition containing the monodisperse particles may comprise an aqueous medium.

As mentioned above, the base coat may comprise a polymer having adhesive properties and/or a coloring agent, especially a black color.

The composition containing the monodisperse particles may be applied after drying the base layer, for example for a period of 30 seconds or longer.

In another of its aspects, the invention provides a method comprising the following steps:
applying a composition comprising hollow monodisperse particles and a medium allowing the formation of an ordered array of monodisperse particles to a support to be made up, which is optionally covered with a base coat; and
applying a top coat to the deposit of the composition containing the catalytic monodisperse particles to improve the hold of the layer of the composition containing the monodisperse particles.

The top coat may comprise a film-forming polymer as mentioned above.

The top coat may be applied after drying the layer of the composition containing the monodisperse particles, for example for a period of 30 seconds or longer.

The invention also provides a method in which a first array of monodisperse particles with an intermediate size is formed, then a second array of monodisperse particles with a mean size that is different from that of the first array is formed on top of said first array.

In another of its aspects, the invention also provides a method comprising the following steps:
applying a first composition comprising hollow monodisperse particles and a medium allowing the formation of an array of said particles; and
applying to the first composition a second composition that can change the color of the first composition, in particular by modifying the refractive index of the medium around the array of particles and/or by modifying the distance between the particles of the array.

In particular, the Applicant has discovered that it is possible to modify in a bespoke manner the coloration obtained by a first cosmetic composition using a second, non colored composition that is subsequently applied.

The crystalline array formed by the first composition may be composed of a continuous layer or discontinuous islets. Light is diffracted by this crystalline array and the wavelength that is diffracted depends on the distance between the particles and the refractive index.

The second composition, which forms the top coat, may contain at least one liquid medium that is capable of penetrating into the first composition and modifying the distance between the particles and/or the refractive index. The liquid medium may optionally be volatile. When it is entirely volatile, the color change is temporary and the color gradually reverts to the initial state. When a large proportion of the liquid medium is non-volatile, a long-lasting color change may be obtained.

The crystalline array may optionally be compact, and may optionally be continuous. It may be formed prior to application or it may be formed during application.

The second composition may contain at least one liquid phase that may swell the array or modify the refractive index of the medium. With a simple change in the refractive index, the liquid phase has a refractive index that is different from the initial medium surrounding the monodisperse particles.

The second composition may also contain a polymer to fix the first composition.

It is possible to use monomers or pre-polymers that are also capable of polymerizing after application to the skin, either by the action of UV, or by heat or the presence of water, for example. Examples that may be mentioned are cyanoacrylate monomers or low mass silicone polymers carrying reactive functions.

A colored or non colored base coat may optionally be applied to the keratinous substances before these two compositions.

In another of its aspects, the invention provides a method in which an array of hollow monodisperse particles is formed on the keratinous substances and a composition is applied to said array to modify the refractive index around the particles of the array, in particular those of the surface layer of the array, to change the color.

### Modes of Application

The composition containing the hollow monodisperse particles and optionally the compositions intended to form the base and top coats may be applied using an applicator, preferably flocked, for example a tip or a flocked foam, or a brush, in particular with fine flexible bristles.

Application may also, for example, be carried out using a foam, felt, spatula, a frit, a brush, a comb, or a woven or nonwoven material.

Application may also be carried out with a finger or by depositing the composition directly onto the support to be treated, for example by spraying or projecting using a piezoelectric device, for example, or by transferring a layer of composition that has already been deposited on an intermediate support.

The composition containing the monodisperse particles may be applied in a thickness that, for example, is in the range 1 μm to 10 μm, preferably in the range 2 μm to 5 μm.

Application of the composition containing the monodisperse particles is carried out, for example, at a density in the range 1 mg/cm² [milligram/square centimeter] to 5 mg/cm².

The array of monodisperse particles that is formed comprises at least six layers of particles, for example, preferably six to 20 layers.

The compound may be applied to the keratinous substances to allow the array of monodisperse particles to form after deposition.

Thus, the composition medium may be formulated so that evaporation of the solvent or solvents it contains is sufficiently slow to leave time for the particles to become ordered and also to limit the risk of disordered agglomeration of the particles before application.

The top coat is, for example applied in a thickness of 0.5 μm to 10 μm. The base layer is, for example, applied in a thickness of 0.5 μm to 10 μm.

The top coat may be applied by spraying.

### Packaging

The composition may be packaged in any receptacle or on any support provided for the purpose.

The composition may be in the form of a kit comprising two compositions packaged in two separate receptacles.

The composition may be in the form of a kit comprising a first receptacle containing the composition comprising the monodisperse particles and a second receptacle containing at least one of the compositions intended to form the base coat and the top coat.

### EXAMPLE

The amounts indicated are amounts by weight.

### Example 1

### Cosmetic Composition

### a) Composition Containing Hollow Monodisperse Particles

| | |
|---|---|
| particles of polystyrene and cross-linked polymethyl | 20% |
| methacrylate (external diameter 290 nm and internal | |
| diameter 200 nm) * | |
| water | 80% |

* SX866(B) sold by JSR (the particles had been concentrated after centrifuging to produce the desired concentration).

Firstly, a base coat was applied, then the composition containing the hollow monodisperse particles was applied. A very brilliant red color was obtained after drying.

Composition of Base Layer:

| | |
|---|---|
| Ultrasol ® 2075 (Ganz Chemical) * | 80% |
| Cab-O-Jet 200 Black Colorant ** | 10% |
| water | 10% |

* acrylate/ammonium methacrylate copolymer in dispersion in water in a concentration of 50% by weight.
** 130 nm carbon black in 20% aqueous dispersion sold by Cabot Corp.

### Example 2

### Cosmetic Composition

### a) Composition Containing Hollow Monodisperse Particles

| | |
|---|---|
| particles of polystyrene (external diameter 2200 nm and | 20% |
| internal diameter 1800 nm)* | |
| Cab-O-Jet 200 Black Colorant** | 10% |
| Ultrasol ® 2075*** | 10% |
| water | 60% |

*SX859(B) sold by JSR with a coefficient of variation of 6.8% (the particles had been concentrated after centrifuging to produce the desired concentration).
**130 nm carbon black in 20% aqueous dispersion sold by Cabot Corp.
***acrylate/ammonium methacrylate copolymer in dispersion in water in a concentration of 50% by weight sold by Ganz Chemical.

Firstly, a base coat was applied, then the composition containing the hollow monodisperse particles was applied. A very nice rainbow effect color was obtained after drying.

Composition of Base Layer:

| | |
|---|---|
| Ultrasol ® 2075 (Ganz Chemical) * | 80% |
| water | 20% |

* acrylate/ammonium methacrylate copolymer in dispersion in water in a concentration of 50% by weight.

Unless otherwise specified, the expression “comprising a” should be construed as being synonymous with “comprising at least one”.

Unless otherwise specified, the expression “in the range” means the limits are included.

1. A cosmetic composition comprising:
a physiologically acceptable medium comprising an aqueous phase; and
hollow monodisperse particles contained in the aqueous phase and capable of forming an ordered array of monodisperse particles after applying the composition to a support.

2. A cosmetic composition comprising:
a physiologically acceptable medium comprising an anhydrous phase; and
hollow monodisperse particles contained in the anhydrous phase and capable of forming an ordered array of monodisperse particles after applying the composition to a support.

3. A composition according to claim 1, the hollow monodisperse particle size being in the range 100 nm to 500 nm.

4. A composition according to claim 3, the hollow monodisperse particle size being in the range 190 μm to 450 mm.

5. A composition according to claim 1, the hollow monodisperse mean particle size being in the range 800 nm to 3000 nm.

6. A composition according to claim 5, the mean particle size being in the range 1200 nm to 2500 nm.

7. A composition according to claim 5, wherein the wall thickness lies in the range 100 nm to 600 nm.

8. A composition according to claim 7, wherein the wall thickness lies in the range 200 nm to 500 nm.

9. A composition according to claim 1, the hollow particles having a wall thickness in the range 10 nm to 300 nm.

10. A composition according to claim 9, the wall thickness being in the range 30 nm to 200 nm.

11. A composition according to claim 10, the wall thickness being in the range 50 nm to 150 nm.

12. A composition according to claim 1, the coefficient of variation (CV) of the size of the monodisperse particles being 5% or less.

13. A composition according to claim 1, in which the hollow monodisperse particles are colored.

14. A composition according to claim 1, the hollow monodisperse particles comprising an inorganic compound.

15. A composition according to claim 1, the hollow monodisperse particles comprising an organic compound.

16. A composition according to claim 15, the hollow monodisperse particles comprising a polymer selected from polystyrene (PS), polymethyl methacrylate (PMMA), polyacrylamide and mixtures and derivatives thereof.

17. A composition according to claim 1, the amount of the hollow monodisperse particles in the composition being 15% by weight or more.

18. A composition according to claim 17, the content being 20% or more.

19. A composition according to claim 17, the amount of the hollow monodisperse particles relative to the phase containing them being 15% by weight or more.

20. A makeup method comprising applying a cosmetic composition as defined in claim 1 to keratinous substances.
